(19) United States
(12) Patent Application Publication
(10) Pub. No.: US 2025/0093255 A1
(43) Pub. Date: Mar. 20, 2025

(54) Patterned Flow Cell and Preparation Method Thereof, and Related Sequencing Method

(71) Applicants: SEQULITE GENOMICS US, INC., Dublin, CA (US); Guangdong Runpeng Biological Technology Co., LTD., Dongguan City (CN)

(72) Inventor: Xiongwei YAN, Dublin, CA (US)

(21) Appl. No.: 18/886,909

(22) Filed: Sep. 16, 2024

Related U.S. Application Data
(60) Provisional application No. 63/538,508, filed on Sep. 15, 2023.

(51) Int. Cl. G01N 15/1434; C12Q 1/6869

(52) U.S. Cl. CPC G01N 15/1436; C12Q 1/6869

(57) ABSTRACT

Provided are a patterned flow cell and a preparation method thereof, and a related sequencing method. The method for preparing a patterned flow cell includes: a) providing a flow cell substrate, wherein the flow cell substrate contains binding regions, and the binding regions are arranged in a patterned manner on the flow cell substrate; b) reacting a reaction reagent with the binding regions to obtain a substrate bound with a linker, wherein the linker contains a halogen atom from the reaction reagent; and c) connecting the substrate bound with the linker with a first nucleic acid to obtain a patterned flow cell.

### CROSS-REFERENCE TO RELATED APPLICATION

This application claims priority to U.S. Provisional Application No. 63/538,508 filed on Sep. 15, 2023, the application of which is hereby incorporated by reference in its entirety.

### TECHNICAL FIELD

The present application relates to the technical field of biology, and in particular, to a patterned flow cell and a preparation method thereof, and a related sequencing method.

### BACKGROUND

In a gene sequencing process, surface modification needs to be performed on micro- and nano-scale array, so as to adsorb an adapter that can capture a nucleic acid molecule to be tested (target nucleic acid molecule). In the prior art, a target region is modified generally with photolithography or printing technology. Further, in conventional sequencing methods, an adapter, a primer, and a DNA fragment are attached to a microsphere, and then the modified microsphere is loaded to a sensor well array for detection.

However, during actual use, such methods are cumbersome in step and low in efficiency, and time costs and economic costs required for preparation of a patterned flow cell are increased, such that it is difficult to meet the needs of large-scale use.

### SUMMARY

The present application is mainly intended to provide a patterned flow cell and a preparation method thereof, and a related sequencing method, so as to solve the problem of cumbersome steps for preparing a patterned flow cell in the prior art.

In order to implement the above objective, a first aspect of the present application provides a method for preparing a patterned flow cell. The method includes: a) a flow cell substrate is provided, wherein the flow cell substrate contains binding regions, and the binding regions are arranged in a patterned manner on the flow cell substrate; b) a reaction reagent is reacted with the binding regions to obtain a substrate bound with a linker, wherein the linker contains a halogen atom from the reaction reagent; and c) the substrate bound is connected with the linker with a first nucleic acid to obtain a patterned flow cell.

Further, the reaction reagent contains a first binding functional group, each of the binding region contains a second binding functional group that is used to react with the first binding functional group, the first binding functional group and the second binding functional group are each independently selected from the group consisting of an active ester group, a sulfonyl halide group, an iodoacetyl group, an alkyne group, an amino group, a thiol group, or an azido group. The reaction reagent further contains a third binding functional group that is used to connect with the first nucleic acid, and the first nucleic acid contains a fourth binding functional group that is used to connect with the third binding functional group; or the reaction reagent contains a halogenated acetyl group that is used to initiate Atom Transfer Radical Polymerization (ATRP). The third binding functional group contains the halogen atom; the third binding functional group and the fourth binding functional group are respectively selected from the sulfonyl halide group and the amino group, or the third binding functional group and the fourth binding functional group are respectively selected from the halogenated acetyl group and the thiol group. The first binding functional group and third binding functional group in the reaction reagent are different.

Further, the method further includes: d) the patterned flow cell is connected with a microsphere after the patterned flow cell is obtained, wherein the microsphere contains a second nucleic acid that is able to bond to the first nucleic acid.

Further, the reaction reagent includes a halogenated acetylation reagent, and the halogenated acetylation reagent contains the halogen atom; and the binding region is the flow cell substrate connected with a first group. The b) includes: the binding region containing the first group is reacted with the halogenated acetylation reagent to form the linker, and the substrate bound with the linker is obtained, wherein the first group has a structure shown in Formula I, and the halogenated acetylation reagent has a structure shown in Formula II.

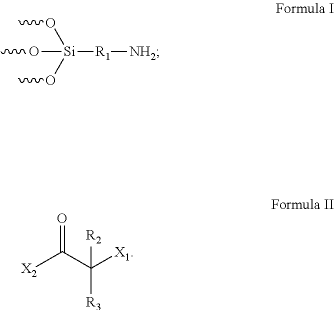

Correspondingly, the linker has a structure shown in Formula III.

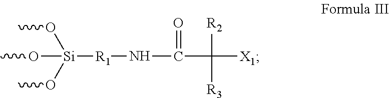

wherein X₁ and X₂ are each independently selected from the halogen atom; Si is a silicon atom, the silicon atom is connected with the flow cell substrate through an oxygen atom, R1 is selected from alkylidene consisting of 1-4 carbon atoms; R2 and R3 are each independently selected from H, alkyl, or aryl. Preferably, the halogen atom is independently selected from Cl, Br, or I. Preferably, the halogenated acetylation reagent includes α-Bromoisobutyryl bromide or Bromoacetyl bromide. Preferably, a method for preparing the binding region includes: a silane is in contact with a partial region on the flow cell substrate to obtain the binding region, wherein the silicon atoms in the first group and the linker are all from the silane. And more preferably, the silane includes (3-Aminopropyl) triethoxysilane (APTES) or 3-Aminopropyl) trimethoxysilane (APTMS).

Further, after the substrate bound with the linker is obtained and before mixing with the first nucleic acid, the substrate bound with the linker is mixed with a polymeric monomer and a catalyst and is subjected to ATRP to obtain a substrate bound with a hydrogel, and then the substrate bound with the hydrogel is bound to the first nucleic acid to obtain the patterned flow cell. Preferably, the polymeric monomer includes methacrylic acid, acrylamide, N,N-dimethylacrylamide, methyl acrylate, or methyl methacrylate. Preferably, the catalyst includes a copper atom; and more preferably, the catalyst includes cuprous bromide.

Further, the c) includes: the first nucleic acid contains the thiol group (—SH), and the thiol group substitutes the halogen atom in the linker to obtain the patterned flow cell.

Further, the reaction reagent includes organic phosphorus compound, and the organic phosphorus compound contains a phosphonic acid group and the halogen atom; and the binding region includes a metallic oxide. The b) includes: the metallic oxide is reacted with the organic phosphorus compound to form the linker, wherein the linker has a structure shown in Formula IV.

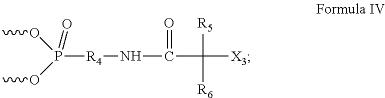

wherein X₃ is the halogen atom; P is a phosphorus atom from the organic phosphorus compound, the phosphorus atom is connected with the metallic oxide through an oxygen atom, R₄ is selected from alkylidene consisting of 1-4 carbon atoms; R₅ and R6 are each independently selected from H, alkyl, or aryl. Preferably, the halogen atom is independently selected from Cl, Br, or I.

Preferably, the organic phosphorus compound includes or

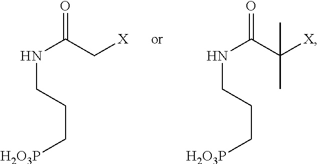

wherein X is Cl, Br, or I.

Further, after the substrate bound with the linker is obtained and before mixing with the first nucleic acid, the substrate bound with the linker is mixed with a polymeric monomer and a catalyst and is subjected to ATRP to obtain a substrate bound with a hydrogel, and then the substrate bound with the hydrogel is bound to the first nucleic acid to obtain the patterned flow cell. Preferably, the polymeric monomer includes methacrylic acid, acrylamide, N,N-dimethylacrylamide, methyl acrylate, or methyl methacrylate. Preferably, the catalyst includes a copper atom; and more preferably, the catalyst includes cuprous bromide.

Further, the c) includes: the first nucleic acid contains the thiol group (—SH), and the thiol group substitutes the halogen atom in the linker to obtain the patterned flow cell.

Further, the metallic oxide includes one or more of aluminum oxide (Al₂O₃), tantalum oxide (Ta₂O₅), niobium oxide (Nb₂O₅), zirconium oxide (ZrO₂) or titanium oxide (TiO₂). Preferably, the flow cell substrate includes a glass material. Preferably, the flow cell substrate includes a bare semiconductor substrate or a semiconductor substrate.

Further, the patterning includes array arrangement. Preferably, the binding region is in a same plane as the flow cell substrate, or there are protrusions or depressions arranged in an array on the flow cell substrate, and the binding region is located in part or all of the protrusions or the depressions.

Further, the d) includes: d1) the microsphere containing the second nucleic acid is mixed with the patterned flow cell, wherein the second nucleic acid is connected with the first nucleic acid through a hydrogen bond formed by complementary base pairing, so as to obtain a hybrid microsphere substrate; and d2) under a light condition or a chemical condition, the second nucleic acid in the obtained hybrid microsphere substrate is connected with the first nucleic acid through a chemical bond, and the patterned flow cell containing the microsphere is obtained after cleaning. Preferably, the light condition includes an ultraviolet radiation.

Further, the microsphere includes an inorganic material microsphere, an organic polymer microsphere, or a DNA Nanoball (DNB). Both the inorganic material microsphere and the organic polymer microsphere are connected to a first target nucleic acid (first nucleic acid to be test) and the second nucleic acids. The DNB includes a second target nucleic acid (second nucleic acid to be test) and the second nucleic acid. Preferably, the inorganic material microsphere includes an alloy, a metallic oxides, or a silicon oxide.

In order to implement the above objective, a second aspect of the present application provides a patterned flow cell. The patterned flow cell is prepared by the method for preparing the patterned flow cell.

In order to implement the above objective, a third aspect of the present application provides a patterned flow cell. A substrate of the patterned flow cell is a flow cell substrate, the flow cell substrate contains connection clusters arranged in an array, each connection cluster contains a plurality of connection chains, each connection chain includes a first nucleic acid and a linker; in a direction away from the substrate of the patterned flow cell, the flow cell substrate, the linker, and the first nucleic acid are connected in sequence.

Further, the patterned flow cell further contains a microsphere. A second nucleic acid is provided on the microsphere, and the second nucleic acid is connected with the first nucleic acid. The second nucleic acid, the first nucleic acid, and the linker form a connection structure, and the microsphere and the connection structure form a microsphere chain, so as to connect the microsphere on the flow cell substrate. The microsphere further contains a target nucleic acid.

Further, the microsphere chain is in a same plane as the flow cell substrate, or there are protrusions or depressions arranged in an array on the flow cell substrate, and the microsphere chain is located in part or all of the protrusions or depressions. Preferably, the microsphere includes an inorganic material microsphere, an organic polymer microsphere, or a DNB). Both the inorganic material microsphere and the organic polymer microsphere are connected to a first target nucleic acids and the second nucleic acids. The DNB includes a second target nucleic acid and the second nucleic acid. Preferably, the inorganic material microsphere includes alloys, metallic oxides, or silicon oxide.

Further, in the connection structure, the linker and the first nucleic acid are directly connected through a chemical bond, or the linker forms a hydrogel and then is connected with the first nucleic acid through the hydrogel. Preferably, the first nucleic acid and the second nucleic acid are connected through a hydrogen bond formed by complementary base pairing, or connected through a chemical bond formed by cross-linking under a light condition or a chemical condition. Preferably, the linker has a structure shown in Formula III. The linker is connected with the flow cell substrate through an oxygen atom.

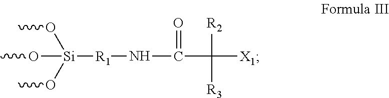

or
the linker has a structure shown in Formula IV, and the linker is connected with a metallic oxide on the flow cell substrate,

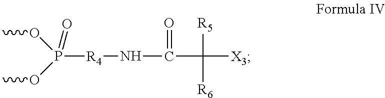

wherein, X₁ or X₃ is each independently selected from a halogen atom, R₁ or R₄ is each independently selected from alkylidene consisting of 1-4 carbon atoms, and R₂, R₃, R₅, or R₆ is each independently selected from H, alkyl, or aryl. Preferably, the halogen atom is independently selected from Cl, Br, or I. Preferably, the flow cell substrate includes a glass material. Preferably, the flow cell substrate includes a bare semiconductor substrate or a semiconductor substrate. Preferably, the metallic oxide includes one or more of aluminum oxide (Al₂O₃), tantalum oxide (Ta₂O₅), niobium oxide (Nb₂O₅), zirconium oxide (ZrO₂) or titanium oxide (TiO₂).

In order to implement the above objective, a fourth aspect of the present application provides a sequencing method. The sequencing method includes sequencing a target nucleic acid (a nucleic acid to be tested) with the patterned flow cell.

Further, the sequencing method includes sequencing by synthesis (SBS). Preferably, the sequencing method includes: S1) a sequencing primer is annealed onto the target nucleic acid in the microsphere, and the sequencing primer is extended with polymerase and nucleoside triphosphate, so as to generate a sequencing signal; and S2) the sequencing signal is analyzed, so as to determine sequences of the target nucleic acid in the plurality of microspheres on the patterned flow cell.

Applying the technical solutions of the present application, in the method for preparing the patterned flow cell, the halogen atoms from the reaction reagent are utilized to connect the first nucleic acid to the flow cell substrate to obtain the patterned flow cell. The method is simple and high in efficiency. The patterned flow cell, obtained through the method, can efficiently and conveniently capture the microspheres during subsequent use to form a loaded array, such that high throughput sequencing can be completed by using same for subsequent sequencing processes.

### BRIEF DESCRIPTION OF THE DRAWINGS

The drawings, which form a part of the present application, are used to provide a further understanding of the present application. The exemplary embodiments of the present application and the description thereof are used to explain the present application, but do not constitute improper limitations to the present application. In the drawings:

FIG. 1 is a schematic diagram of a method for preparing a patterned flow cell according to a first embodiment of the present application.

FIG. 2 is a schematic diagram of a method for preparing a patterned flow cell according to a second embodiment of the present application.

FIG. 3 is a schematic diagram of a method for preparing a patterned flow cell according to a third embodiment of the present application.

FIG. 4 is a schematic diagram of a method for preparing a patterned flow cell according to a fourth embodiment of the present application.

FIG. 5 is a diagram of an imaging result of a patterned flow cell according to Example 1 of the present application.

FIG. 6 is a reaction principle diagram of preparation of hydrogel according to Example 2 of the present application.

FIG. 7 is a diagram of an imaging result of a patterned flow cell according to Example 2 of the present application.

FIG. 8 is a diagram of an imaging result of a sequencer according to Example 2 of the present application.

FIG. 9 is a reaction principle diagram of preparation of hydrogel according to Example 3 of the present application.

### DETAILED DESCRIPTION OF THE EMBODIMENTS

It is to be noted that the embodiments in the present application and the features in the embodiments may be combined with one another without conflict. The present application will be described below in detail with reference to the embodiments.

### Definitions

It should be understood that terms used herein will be understood to have their ordinary meaning in the relevant field unless otherwise indicated. A number of terms used herein and meanings thereof are listed below.

The singular forms “a”, “an” and “the” include plural referents unless the context clearly indicates otherwise.

The terms “contain”, “include”, “comprise”, “accommodate”, and the various forms of these terms are synonymous with each other and are intended to be equally broad.

The terms first, second, etc. are also not intended to imply a particular orientation or order, but are used to distinguish one component from another. “A first target nucleic acid” and “a second target nucleic acid” in the present application are only intended to distinguish the target nucleic acids contained on different microspheres.

As used herein, “alkyl” refers to a straight or branched hydrocarbon chain that is fully saturated (i.e., not containing a double bond or a triple bond). An alkyl group may have 1 to 20 carbon atoms (including but not limited to 1, 2, 3, 4, 5, 6, 7, 8, 9, 10, 11, 12, 13, 14, 15, 16, 17, 18, 19 or 20 carbon atoms). A typical alkyl group includes methyl, ethyl, propyl, isopropyl, butyl, isobutyl, tert butyl, pentyl, hexyl, and the like. As an example, the name “C1-C4 alkyl” indicates that there are one to four carbon atoms in an alkyl chain, that is, the alkyl chain is selected from methyl, ethyl, propyl, isopropyl, n-butyl, isobutyl, sec-butyl, and tert-butyl.

As used here, “alkylidene” refers to a bivalent straight or branched hydrocarbon chain that is fully saturated (i.e., not containing a double bond or a triple bond). The name “alkylidene consisting of 1-4 carbon atoms” indicates that there are one to four carbon atoms in the alkylidene, that is, the alkylidene is selected from methylene (—CH₂—), ethylidene (—CH₂—CH₂—), propylidene (—CH₂—CH₂—CH₂—), isopropylidene

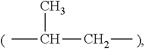

butylene (—CH₂—CH₂—CH₂—CH₂—), isobutylidene

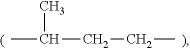

sec-butylidene

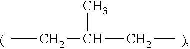

and tert-butylidene

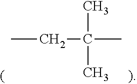

As used here, “aryl” refers to an aromatic ring or a ring system only containing carbon in a ring skeleton (i.e., two or more condensed rings sharing two adjacent carbon atoms). When the aryl is the ring system, each ring in the ring system is aromatic. An aryl group may have 6 to 18 carbon atoms. An example of the aryl group includes phenyl, naphthyl, an azulenyl, and anthryl.

As used here, “nucleotide” includes a nitrogen-containing heterocyclic base, sugar, and one or more phosphate groups. The nucleotide is a monomer unit of a nucleic acid sequence. In RNA, the sugar is ribose. And in DNA, the sugar is deoxyribose, that is, sugar of a hydroxyl group at Position 2′ is absent in the ribose. The nitrogen-containing heterocyclic base (i.e., nucleobase) may be a purine base or a pyrimidine base. The purine base includes Adenosine (A), Guanine (G), and modified derivatives or analogues thereof. The pyrimidine base includes Cytosine (C), Thymine (T), Uracil (U), and modified derivatives or analogues thereof. A C-1 atom of the deoxyribose is bonded to N-1 of pyrimidine or N-9 of purine. A nucleic acid analogue may have any one of a modified phosphate backbone, sugar, or nucleobase. An example of the nucleic acid analogue includes, for example, a universal base or a phosphate-sugar backbone analogue, such as Peptide Nucleic Acid (PNA).

“Patterned flow cell” refers to a single-layer substrate including patterned surface chemical compositions, or a multi-layer stack having layers including the patterned surface chemical compositions. The pattern is, for example, in a depression portion. The surface chemical compositions may include polymer hydrogel and nucleic acids, and may be used to capture and amplify microspheres, for example.

The term “flow cell substrate” refers to a single-layer or multi-layer structure on which surface chemicals are introduced or no surface chemicals are contained.

The term “halogen” or “halogen atom” includes bromine, chlorine, fluorine, or iodine.

The term “hydrogel” is a polymer material having a three-dimensional cross-linked network structure, which has high water absorbability. The hydrogel mainly consists of hydrophilic polymer chains, and these polymer chains are cross-linked together in a chemical or physical manner, so as to form a porous structure.

The term “ATRP” or “Atom Transfer Radical Polymerization” is a method for controlling free radical polymerization, which may obtain polymers with accurate molecular weights, narrow molecular weight distribution and tunable functions. When the ATRP is used for producing the hydrogel, some specific functional monomers are generally involved. These monomers may be bound with water molecules in a chemical or physical manner to form a three-dimensional network structure, so as to form the hydrogel. Principles are as follows:

Initiation: polymerization starts through an appropriate initiator (e.g., halogenated alkane) to generate an active free radical.

Propagation: the active free radical and a monomer (e.g., water-soluble monomer) are reacted to form a propagating chain.

Atom transfer: a metal catalyst (e.g., copper or iron complexes) is reacted with a terminal of the propagating chain to form a dormant species. This process reduces the concentration of the active free radical, so as to control a polymerization rate.

Chain transfer: the dormant species may be re-activated through halogen atom transfer of another halogenated alkane molecule to generate a new active free radical, so as to continue a polymerization process.

Cross-linking: during the polymerization process, if a monomer containing two or more reactive functional groups is used, these functional groups may form covalent bonds, so as to form the three-dimensional network structure.

As used here, the term “depression” refers to a discrete concave feature having a surface opening in the flow cell substrate, wherein the surface opening is at least partially surrounded by gap regions in the flow cell substrate. A depression portion may have any one among various shapes at the opening in the surface, including, for example, a circle, an oval, a square, a polygon, a star (having any number of vertices), etc. A cross section of the depression portion intercepted orthogonally to the surface may be curved, square, polygonal, hyperbolic, conical, angular, or the like. For example, the depression portion may be a hole or two interconnected holes. The depression portion may also have a more complex structure, such as a ridge, a step feature portion, and the like.

As used here, the term “protrusion” refers to a discrete convex feature in the flow cell substrate, wherein the protrusion is at least partially surrounded by the gap regions in the flow cell substrate. A protrusion portion may have any one among various shapes at the protrusion in the surface, including, for example, a circle, an oval, a square, a polygon, a star (having any number of vertices) etc. A cross section of the protrusion portion intercepted orthogonally to the surface may be curved, square, polygonal, hyperbolic, conical, angular, or the like. For example, the protrusion portion may be a columnar structure or a platform-like structure. The protrusion portion may also have a more complex structure, such as a ridge, a step feature portion, a wave, or the like.

As used here, the term “microsphere” includes, but is not limited to, a spherical particle formed by an inorganic material, an organic polymer material, or DNA, and plays the same or similar role as the “microsphere” used in high throughput sequencing in the prior art. A diameter of the microsphere is preferably 1 nm-100 m, and more preferably 100 nm-100 m.

As used here, the term “array arrangement” refers to a structure that is formed through the arrangement of binding regions (or microsphere clusters) in a certain rule and sequence in a top view of the patterned flow cell. Such arrangement is usually regular in nature and may be linear, grid-like, spiral or other geometric. The binding regions (or microsphere clusters) arranged in an array may improve the efficiency and flux of an experiment. As they allow a plurality of experiments to be performed simultaneously, the consumption of samples and reagents is reduced, and an automated processing procedure is simplified. Furthermore, the arrangement mode also facilitates the standardization and comparison of data as it ensures the consistency of all experimental conditions.

As mentioned in the Background, the step of performing surface modification on micro- and nano-scale arrays and the subsequent steps of library preparation and loading of the microspheres into the arrays in the prior art are cumbersome, thus increasing the cost of the sequencing process. Therefore, the inventor in the present application has attempted to develop a method for preparing a patterned flow cell with simple steps and high efficiency. With the method, the patterned flow cell containing a first nucleic acid is rapidly and efficiently prepared from the flow cell substrate with a reaction reagent containing a halogen atom in the method, such that the patterned flow cell can be rapidly and conveniently bound with the microspheres in subsequent applications, and applications such as sequencing are achieved. Based on the method, a series of protection methods of the present application are proposed.

A first typical implementation of the present application provides a method for preparing a patterned flow cell. The method includes: a) a flow cell substrate is provided, wherein the flow cell substrate contains binding regions, and the binding regions are arranged in a patterned manner on the flow cell substrate; b) a reaction reagent is reacted with the binding regions to obtain a substrate bound with a linker, wherein the linker contains a halogen atom from the reaction reagent; and c) the substrate bound is connected with the linker with a first nucleic acid to obtain the patterned flow cell.

In the method, the patterned flow cell is obtained by connecting the linker and the first nucleic acid to partial regions (i.e., the binding regions) on the flow cell substrate. In the method, the flow cell substrate is provided first, partial region on the flow cell substrate contains the binding regions, and subsequent groups are all reacted at the positions of the binding regions to obtain the final patterned flow cell. Therefore, patterns on the patterned flow cell are controlled by the positions of the binding regions.

Secondly, the reaction reagent containing the halogen atom is reacted with the binding regions, partial groups in the reaction reagent are connected to the binding regions, and the flow cell substrate is currently prepared to the substrate bound with the linker.

Further, the substrate bound with the linker is mixed with the first nucleic acid, the first nucleic acid is bond with the linker on the substrate, and such binding includes, but is not limited to, a connection by means of a physical adsorption, a hydrogen bond, a chemical bond, or other manners, so as to obtain the patterned flow cell.

In the method, the used flow cell substrate and the binding regions may be located in the same plane (2-D structure), or may also be located in different planes (3-D structure). For example, the flow cell substrate contains protrusions or depressions, the binding regions are located on surfaces of the protrusions or depressions, and the surfaces (or cross-sections) of the protrusions or depressions may be parallel to or intersect with a surface of the flow cell substrate. By controlling settings such as sizes, positions, and arrangement of the binding regions, the patterns in the patterned flow cell subsequently obtained through the method are controlled.

In a preferred embodiment, the reaction reagent contains a first binding functional group, each of the binding regions contains a second binding functional group that is used to react with the first binding functional group. The first binding functional group and the second binding functional group are each independently selected from the group consisting of an active ester group, a sulfonyl halide group, an iodoacetyl group, an alkyne group, an amino group, a thiol group, or an azido group.

In the method, through the reaction between the first binding functional group and the second binding functional group, the reaction reagent is connected to the binding region, so as to obtain the substrate bound with the linker. The reaction during this process includes, but is not limited to, a reaction between the active ester group and the amino group, a reaction between the sulfonyl halide group and the thiol group, a reaction between the alkynyl group and the azido group, etc. Based on the above chemical reactions in the prior art, the first binding functional group and the second binding functional group are flexibly set, such that the proceeding of the step b) in the method can be guaranteed.

In a preferred embodiment, the reaction reagent also contains a third binding functional group that is used to connect with the first nucleic acid, and the first nucleic acid contains a fourth binding functional group that is used to connect with the third binding functional group; or the reaction reagent contains a halogenated acetyl group that is used to initiate ATRP; and the third binding functional group and the fourth binding functional group are each independently selected from an active ester group, a sulfonyl halide group

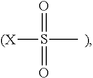

an iodoacetyl group

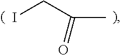

an alkyne group, an amino group, a thiol group, or an azido group.

In the step c) of the method, the substrate bound with the linker is connected with the first nucleic acid, and such connection includes a connection that is realized with the chemical reaction between the third binding functional group and the fourth binding functional group. The third binding functional group is derived from the reaction reagent, is bound with the flow cell substrate through the step b), and plays a role in the step c). The fourth functional group may be flexibly arranged on a 5′ end or 3′ end of the first nucleic acid, or inside the sequence of the first nucleic acid.

Optionally, in another mode of the step c), with the halogenated acetyl group (which is derived from the reaction reagent, and bound with the flow cell substrate through the step b)) that can initiate ATRP, the hydrogel is prepared by initiating polymerization or copolymerization on the flow cell substrate, and then the capturing and connection of the first nucleic acid are realized through the hydrogel.

The active ester group is a group containing an ester group (—COOR), wherein a carbon atom of the ester group is connected with a good leaving group (e.g., a chlorine atom, a sulfate radical, a phosphate radical, etc.). Through such structure, it is easier for the active ester group to lose the leaving group in the chemical reaction to generate a negatively-charged ester group, so as to participate in various chemical reactions such as transesterification, amidation, and the like. The active ester includes, but is not limited to, chloroformate (—COCl), sulfate (—O—SO₃H), or phosphate (—O—PO₃H₂).

Preferably, the first binding functional group and the third binding functional group in the reaction reagent are different.

In order to guarantee the specificity of the reaction, the first binding functional group and the third binding functional group are different groups, and cannot both react with the second binding functional group. Such design can ensure that only the reaction between the first binding functional group and the second binding functional group is underwent in the step b), and the third binding functional group does not undergo a chemical reaction in the step b).

Preferably, the reaction reagent has structures such as

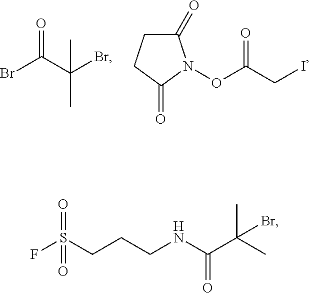

and the like.

In a preferred embodiment, the method further includes: d) the patterned flow cell is connected with a microsphere after the patterned flow cell is obtained, wherein the microsphere contains a second nucleic acid that is able to bond to the first nucleic acid.

In the step d), the microsphere containing the second nucleic acid is mixed with the patterned flow cell, and the first nucleic acid and the second nucleic acid can undergo at least partial sequence binding, such that the microsphere is connected with the flow cell substrate to obtain the patterned flow cell.

In a preferred embodiment, the reaction reagent includes a halogenated acetylation reagent, and the halogenated acetylation reagent contains the halogen atom; and the binding region is the flow cell substrate connected with a first group. The b) includes: the binding region containing the first group is reacted with the halogenated acetylation reagent to form the linker, and the substrate bound with the linker is obtained, wherein the first group has a structure shown in Formula I, and the halogenated acetylation reagent has a structure shown in Formula II; and correspondingly, the linker has a structure shown in Formula III.

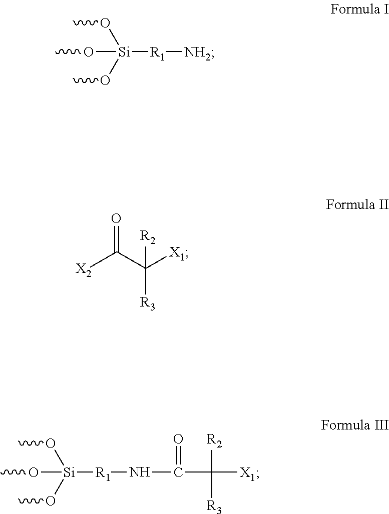

wherein X₁ and X₂ are each independently selected from the halogen atom; Si is a silicon atom, the silicon atom is connected with the flow cell substrate through an oxygen atom, R₁ is selected from alkylidene consisting of 1-4 carbon atoms; R₂ and R₃ are each independently selected from H, alkyl, or aryl. Preferably, the halogen atom is independently selected from Cl, Br, or I. Preferably, the halogenated acetylation reagent includes α-Bromoisobutyryl bromide

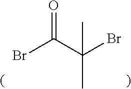

or Bromoacetyl bromide

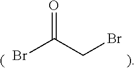

Preferably, a method for preparing the binding region includes: silane is in contact with partial region on the flow cell substrate to obtain the binding region, wherein the silicon atoms in the first group and the linker are all from the silane. More preferably, the silane includes (3-Aminopropyl) triethoxysilane

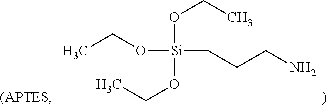

### or 3-Aminopropyl)trimethoxysilane

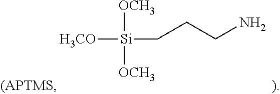 [SMILES: CO[Si](CCCN)(OC)OC]

In the method, the reaction with the first group on the binding region is performed with the halogenated acetylation reagent, high reaction efficiency is realized, and the reaction is almost quantitatively completed (a conversion rate being close to 100%).

In the method, on the binding region of the flow cell substrate used, the first group is connected with the flow cell substrate through the oxygen atom, the first group undergoes the reaction (haloacetylation) after being in contact with the halogenated acetylation reagent to generate the linker shown in Formula III and a compound HX₂. The linker can directly react with a group on a nucleic acid, so as to realize the connection of the nucleic acid (oligonucleotide conjugation). Or can also be used as an initiator in ATRP, which is mixed with a polymeric monomer and a catalyst, then undergoes ATRP to prepare the hydrogel. And is bound with the nucleic acid to realize a connection with the hydrogel.

A person skilled in the art may flexibly select the polymeric monomer and the catalyst according to known principles and methods of ATRP in the prior art. Preferably, the polymeric monomer includes, but is not limited to, methacrylic acid, acrylamide, N,N-dimethylacrylamide, methyl acrylate, or methyl methacrylate. Preferably, the catalyst contains a copper element, including but not limited to cuprous bromide.

In a preferred embodiment, after the substrate bound with the linker is obtained and before mixing with the first nucleic acid, the substrate bound with the linker is mixed with a polymeric monomer and a catalyst and is subjected to ATRP to obtain a substrate bound with hydrogel (hydrogel formation). And then the substrate bound with hydrogel is bound to the first nucleic acid to obtain the patterned flow cell. Preferably, the polymeric monomer includes methacrylic acid, acrylamide, N, N-dimethylacrylamide, methyl acrylate, or methyl methacrylate. Preferably, the catalyst includes a copper atom; and more preferably, the catalyst includes cuprous bromide.

The principle of binding the first nucleic acid with the hydrogel includes, but is not limited to, any one or a combination of the following:
1. Physical adsorption: the hydrogel fixes the nucleic acid on a surface or inside the hydrogel by means of physical adsorption.
2. Covalent linkage: the nucleic acid is covalently linked with a polymer substrate of the hydrogel by means of a chemical method.
3. Electrostatic interaction: there may be an electrostatic interaction between the nucleic acid and the hydrogel, and such interaction may promote the binding of the nucleic acid with the hydrogel. For example, a negatively-charged phosphate group in a nucleic acid molecule may be bound with a positive-charged hydrogel by means of electrostatic attraction.
4. Hydrophobic interaction: a hydrophobic group in the nucleic acid molecule may interact with a hydrophobic region in the hydrogel, so as to realize the binding of the nucleic acid and the hydrogel.
5. Metal ion bridging: metal ions such as Ca²⁺, Mg²⁺, and the like may form complexes with ligands in the nucleic acid and hydrogel, so as to realize the binding of the nucleic acid and the hydrogel.

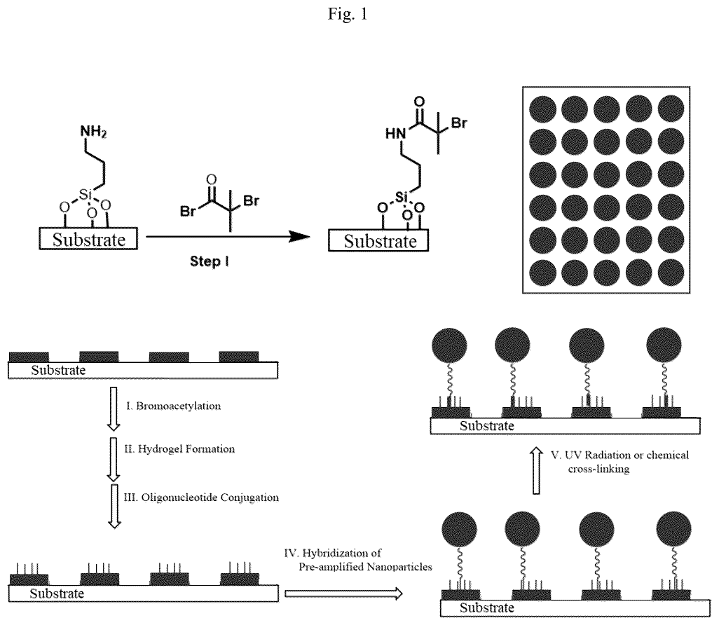

A schematic diagram of the method using the halogenated acetylation reagent to react with the binding region and prepare the hydrogel is shown in FIG. 1.

In a preferred embodiment, the c) includes: the first nucleic acid contains the thiol group (—SH), and the thiol group substitutes the halogen atom in the linker to obtain the patterned flow cell.

If the first nucleic acid contains a group that can react with the linker, such as nucleotide with thio modification, the first nucleic acid can be subjected to a chemical reaction with the linker, a sulfur atom in the nucleic acid can substitute the halogen atom, and connect the first nucleic acid to the flow cell substrate.

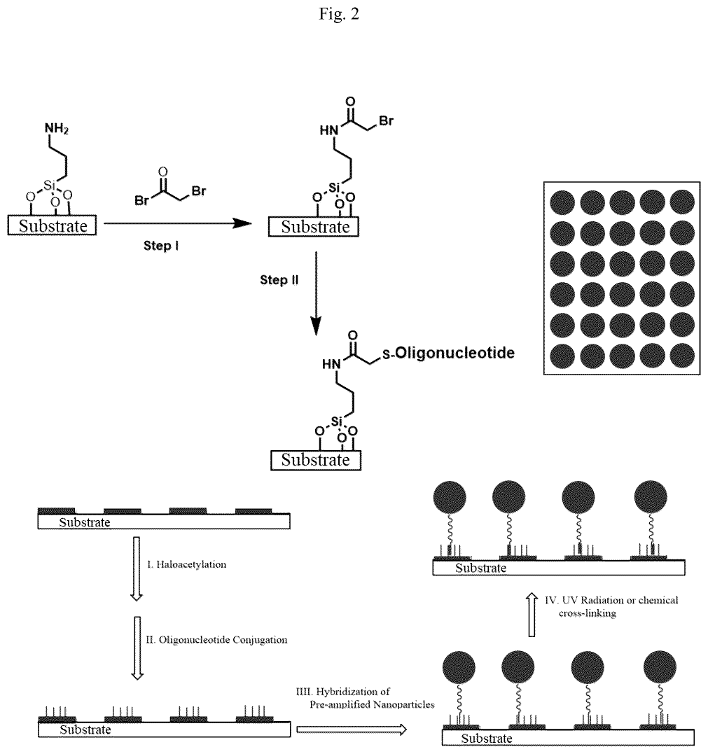

A schematic diagram of the method using the halogenated acetylation reagent to react with the binding region and achieve direct connection with the first nucleic acid is shown in FIG. 2.

If the first nucleic acid is directly subjected to a reaction with the linker, it may be understood that the first nucleic acid is attached to a two-dimensional plane. If the hydrogel is produced on the flow cell substrate before the connection of the first nucleic acid, the nucleic acid can be attached to the hydrogel having a three-dimensional space. In this three-dimensional structure, more first nucleic acids can be attached, making it easier, faster, and stronger to crosslink the second nucleic acid and capture nanoparticles. In such a three-dimensional structure, more first nucleic acids can be attached, making it easier, faster, and stronger to crosslink the second nucleic acid and capture nanoparticles. Moreover, the hydrogel can provide protection for subsequently bound nanoparticles, and prevent the nanoparticles from being damaged due to reasons such as liquid flow during the subsequent use process of the patterned flow cell. The nanoparticles located in the hydrogel can produce complementary second chains more quickly and more easily by means of DNA polymerization during the subsequent use of the patterned flow cell and during PE sequencing, so as to perform subsequent sequencing processes.

In another optional embodiment, the reaction reagent includes organic phosphorus compound, and the organic phosphorus compound contains a phosphonic acid group and the halogen atom; and the binding region includes a metallic oxide. The b) includes: the metallic oxide is reacted with the organic phosphorus compound to form the linker, wherein the linker has a structure shown in Formula IV.

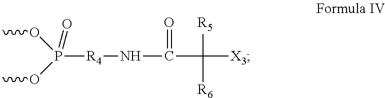

wherein X₃ is the halogen atom; P is a phosphorus atom from the organic phosphorus compound, the phosphorus atom is connected with the metallic oxide through an oxygen atom, R₄ is selected from alkylidene consisting of 1-4 carbon atoms; R₅ and R₆ are each independently selected from H, alkyl, or aryl. Preferably, the organic phosphorus compound includes

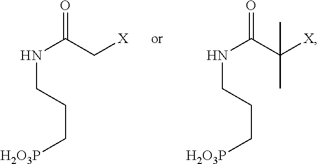

wherein X is Cl, Br, or I.

In another method, the reaction reagent includes the organic phosphorus compound containing a phosphonic acid group (—PO(OH)₂ or —PO₃H₂) and the halogen atom, and the binding region includes the metallic oxide. The metallic oxide on the flow cell substrate are available in forms including, but not limited to, as films, patches, doped compounds, and the like.

A method for setting the metallic oxide on the flow cell substrate includes, but is not limited to:
1. Chemical Vapor Deposition (CVD): gas of a metal organic compound or a metal halide is introduced into a reaction chamber, and then a chemical reaction is performed on the surface of the flow cell substrate to produce the metallic oxide.
2. Physical Vapor Deposition (PVD): with technologies including sputtering, evaporation, ion plating, and the like, the metallic oxide is deposited on the surface of the flow cell substrate through a physical process.
3. Sol-gel method: sol or gel is formed by dissolving a precursor such as metal alkoxide into water, then is coated on the surface of the flow cell substrate, and finally forms a metallic oxide layer through heat treatment.
4. Spray pyrolysis: a metallic oxide precursor is dissolved in a solvent to form a solution, then the solution is sprayed to the surface of the heated flow cell substrate by a sprayer, and after the solvent is evaporated, the metallic oxide is deposited on the flow cell substrate.
5. Electrochemical deposition (ECD): a current is applied to the surface of the flow cell substrate, and metal ions are reduced on an electrode surface to form a metallic oxide film.
6. Thermal spraying: metal powder or metallic oxide powder is melted through heating using high-speed flame or plasma, and then is sprayed to the surface of the flow cell substrate to form a coating.
7. Lithography technology: the surface of the flow cell substrate is coated with a photoresist, partial region is exposed and cured, the photoresist on unexposed regions is removed, and the metallic oxide is arranged on the unexposed regions by means of methods such as PVD, CVD, ALD (Atomic Layer Deposition), or the like, so as to finally remove the cured photoresist.

The phosphonic acid group on the organic phosphorus compound can react with the metallic oxide, and the principle of the chemical reaction is shown as follows:
i) First, the metallic oxide (MO) reacts with water to produce corresponding metal hydroxide (M(OH)n):
MO+nH₂O→M(OH)n; and
ii) Then, the metal hydroxide reacts with the phosphonic acid group to produce metal phosphonate (MPO₃H₂) and water:

M(OH)n+2nPO3H₂→M(PO₃H₂)₂ₙ+2nH₂O.

In the method, with the above chemical reaction, the organic phosphorus compound can be connected with the flow cell substrate to obtain the linker. The linker can directly react with a group on a nucleic acid, so as to realize the connection of the nucleic acid, or can also be used as an initiator in ATRP, which is mixed with a polymeric monomer and a catalyst, then undergoes ATRP to prepare the hydrogel, and the hydrogel is bound with the nucleic acid to realize a connection.

In a preferred embodiment, after the substrate bound with the linker is obtained and before mixing with the first nucleic acid, the substrate bound with the linker is mixed with a polymeric monomer and a catalyst and is subjected to ATRP to obtain a substrate bound with a hydrogel, and then the substrate bound with the hydrogel is bound to the first nucleic acid to obtain the patterned flow cell. Preferably, the polymeric monomer includes methacrylic acid, acrylamide, N,N-dimethylacrylamide, methyl acrylate, or methyl methacrylate. Preferably, the catalyst includes a copper atom; and more preferably, the catalyst includes cuprous bromide.

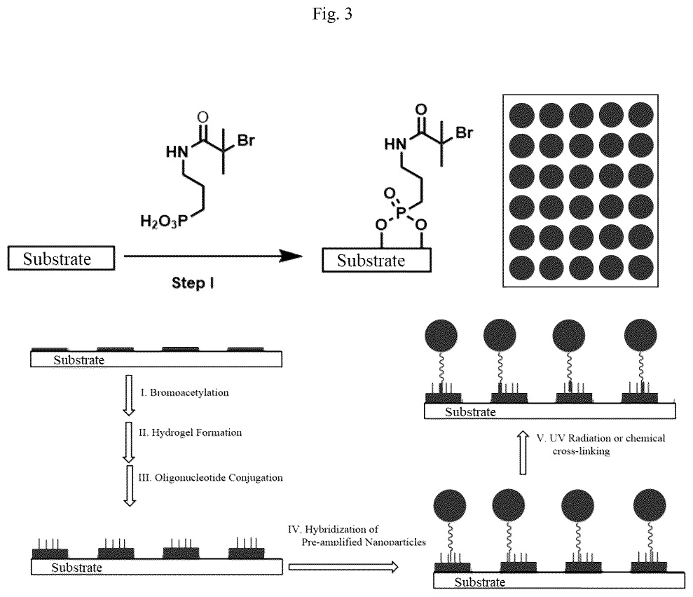

A schematic diagram of the method using the organic phosphorus compound to react with the binding region and prepare the hydrogel is shown in FIG. 3.

In a preferred embodiment, the c) includes: the first nucleic acid contains the thiol group (—SH), and the thiol group substitutes the halogen atom in the linker to obtain the patterned flow cell.

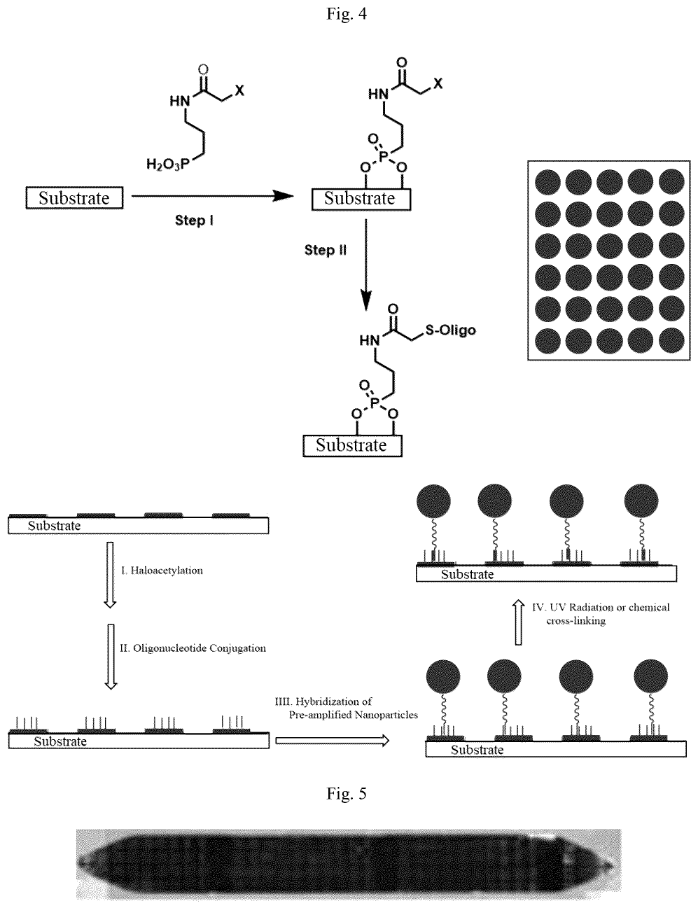

A schematic diagram of the method using the organic phosphorus compound to react with the binding region and then achieve direct connection with the first nucleic acid is shown in FIG. 4.

Similar to the reaction between the halogenated acetylation reagent and the binding region, the organic phosphorus compound is used to react with the binding region, and the produced linker can also be connected with the first nucleic acid after the hydrogel is obtained through preparation, or be directly connected with the first nucleic acid.

In a preferred embodiment, the metallic oxide includes one or more of aluminum oxide (Al₂O₃), tantalum oxide (Ta₂O₅), niobium oxide (Nb₂O₅), zirconium oxide (ZrO₂) or titanium oxide (TiO₂). Preferably, the flow cell substrate includes a glass material. Preferably, the flow cell substrate includes a bare semiconductor substrate or a semiconductor substrate.

In a preferred embodiment, patterning includes array arrangement. Preferably, the binding region is in a same plane as the flow cell substrate, or there are protrusions or depressions arranged in an array on the flow cell substrate, and the binding region is located in part or all of the protrusions or the depressions.

In a preferred embodiment, the d) includes: d1) the microsphere containing the second nucleic acid is mixed with the patterned flow cell, wherein the second nucleic acid is connected with the first nucleic acid through a hydrogen bond formed by complementary base pairing, so as to obtain a hybrid microsphere substrate (hybridization of pre-amplified nonoparticles); and d2) under a light condition or a chemical condition, the second nucleic acid in the obtained hybrid microsphere substrate is connected with the first nucleic acid through a chemical bond, and the patterned flow cell containing the microsphere is obtained after cleaning (UV radiation or chemical cross-linking). Preferably, the light condition includes an ultraviolet radiation. Preferably, a wavelength of an ultraviolet light is 200-400 nm.

In a preferred embodiment, the microsphere includes an inorganic material microsphere, an organic polymer microsphere, or a DNB. Both the inorganic material microsphere and the organic polymer microsphere are connected to the first target nucleic acid and the second nucleic acids. The DNB includes a second target nucleic acid and the second nucleic acid. Preferably, the inorganic material microsphere includes an alloy, a metallic oxide, an organic polymer, or a silicon oxide. More preferably, metal in the inorganic material microsphere is selected from silver, copper, gold or platinum, which is commonly used in the prior art for sequencing.

The microspheres are connected in the patterned flow cell, the microspheres all contain nucleic acids to be tested (target nucleic acids, contain the first target nucleic acid or second target nucleic acid, and the nucleic acids are the same or different), and during the subsequent use of the patterned flow cell and sequencing, base sequence arrangement of the target nucleic acids can be detected by means of methods such as SBS sequencing. After sequencing is completed, polymerase is used to synthesize complementary nucleic acids to be tested, the sequenced target nucleic acids are removed by a hydrolytic enzyme, and the base sequence arrangement of the complementary nucleic acids to be tested can be detected by means of the same methods such as SBS sequencing.

The microspheres further contain second nucleic acids, which are used to bind with the first nucleic acids so as to connect the microspheres on the flow cell substrate.

If the microspheres are metal particles, during the subsequent use of the patterned flow cell and during sequencing, the strength of a sequencing signal can be enhanced to improve a difference between signals of different nucleotides, so as to improve the sensitivity and accuracy of sequencing.

In the method for preparing a patterned flow cell, by introducing the microspheres and the target nucleic acids, the patterned flow cell that can be used for sequencing can be directly prepared in a preparation process, such that the binding efficiency of the target nucleic acids and the patterned flow cell is improved, and test steps and costs required are reduced, thereby improving overall test efficiency.

A second typical implementation of the present application provides a patterned flow cell. The patterned flow cell is prepared by the method for preparing the patterned flow cell.

A third typical implementation of the present application provides a patterned flow cell. A substrate of the patterned flow cell is a flow cell substrate, the flow cell substrate contains connection clusters arranged in an array, each connection cluster contains a plurality of connection chains, each connection chain includes a first nucleic acid and a linker; in a direction away from the substrate of the patterned flow cell, the flow cell substrate, the linker, and the first nucleic acid are connected in sequence.

In a preferred embodiment, the patterned flow cell further contains a microsphere. A second nucleic acid is provided on the microsphere, and the second nucleic acid is connected with the first nucleic acid. The second nucleic acid, the first nucleic acid, and the linker form a connection structure, and the microsphere and the connection structure form a microsphere chain, so as to connect the microsphere on the flow cell substrate. The microsphere further contains a target nucleic acid (a nucleic acid to be tested).

In a preferred embodiment, the microsphere chain is in a same plane as the flow cell substrate, or there are protrusions or depressions arranged in an array on the flow cell substrate, and the microsphere chain is located in part or all of the protrusions or the depressions. Preferably, the microsphere includes an inorganic material microsphere, an organic polymer microsphere, or a DNB. Both the inorganic material microsphere and the organic polymer microsphere are connected to a first target nucleic acid and the second nucleic acid. The DNB includes a second target nucleic acid and the second nucleic acid. Preferably, the inorganic material microsphere includes an alloy, a metallic oxide, or a silicon oxide. More preferably, metal in the inorganic material microsphere is selected from silver, copper, gold or platinum, which is commonly used in the prior art for sequencing.

In the patterned flow cell, the flow cell substrate contains microsphere clusters, each microsphere cluster contain a plurality of microsphere chains, and the microsphere clusters are arranged in an array to form the patterns on the patterned flow cell.

Preferably, a connection point between the microsphere cluster and the flow cell substrate is named as Point A, and a point in the flow cell substrate that is not connected with the microsphere cluster is named as Point B. Point A and Point B may be located in the same plane (2-D structure). Currently, if the microsphere cluster does not contain a hydrogel structure, the microsphere chain is considered to be in the same plane as the flow cell substrate; and if the microsphere cluster contains the hydrogel structure, the microsphere chain is considered to not be in the same plane as the flow cell substrate.

Point A and Point B may also be located in different planes (3-D structure). For example, the flow cell substrate contains protrusions or depressions, Point A is located on surfaces (including but not limited to planes or sidewalls formed by the protrusions or depressions) of the protrusions or depressions, and the surfaces (or cross sections) of the protrusions or depressions may be parallel to or intersect with the surface of the flow cell substrate.

The patterned flow cell contains the microspheres, the microspheres contain the nucleic acids to be tested (the first target nucleic acid or second target nucleic acid), and during the subsequent use of the patterned flow cell and sequencing, base sequence of the target nucleic acids can be detected by means of methods such as SBS sequencing.

If the microspheres are metal particles, during the subsequent use of the patterned flow cell and during sequencing, the strength of a sequencing signal can be enhanced to improve a difference between signals of different nucleotides, so as to improve the sensitivity and accuracy of sequencing.

In a preferred embodiment, in the connection structure, the linker and the first nucleic acid are directly connected through a chemical bond, or the linker forms a hydrogel and then is connected with the first nucleic acid through the hydrogel.

In a preferred embodiment, the first nucleic acid and the second nucleic acid are connected through a hydrogen bond formed by complementary base pairing, or connected through a chemical bond formed by cross-linking under a light condition or a chemical condition.

In a preferred embodiment, the linker has a structure shown in Formula III,

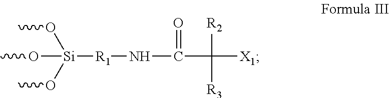

the linker is connected with the flow cell substrate through an oxygen atom; or
the linker has a structure shown in Formula IV, and the linker is connected with a metallic oxide on the flow cell substrate,

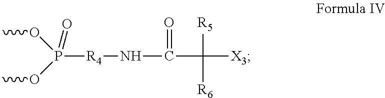

X₁ or X₃ is each independently selected from a halogen atom, R₁ or R₄ is each independently selected from alkylidene consisting of 1-4 carbon atoms, and R₂, R₃, R₅, or R₆ is each independently selected from H, alkyl, or aryl. Preferably, the halogen atom is independently selected from Cl, Br, or I. Preferably, the flow cell substrate includes a glass material. Preferably, the flow cell substrate includes a bare semiconductor substrate or a semiconductor substrate.

In a preferred embodiment, the metallic oxide includes one or more of aluminum oxide (Al₂O₃), tantalum oxide (Ta₂O₅), niobium oxide (Nb₂O₅), zirconium oxide (ZrO₂) or titanium oxide (TiO₂).

A fourth typical implementation of the present application provides a sequencing method. The sequencing method includes sequencing the target nucleic acids with the patterned flow cell or the patterned flow cell prepared by the method.

In a preferred embodiment, the sequencing method includes sequencing by synthesis (SBS).

In a preferred embodiment, the sequencing method includes: S1) a sequencing primer is annealed onto the target nucleic acid in a microsphere, the sequencing primer is extended with a polymerase and a nucleoside triphosphate, so as to generate a sequencing product, and the nucleoside triphosphate is added to a 3′ end of the sequencing primer, so as to generate a sequencing signal; and S2) the sequencing signal is analyzed, so as to determine sequences of the target nucleic acids in the plurality of microspheres on the patterned flow cell.

The beneficial effects of the present application are further described in detail below with reference to specific embodiments.

### Example 1

i. Prewash APTES functionalized surface (flow cells) with 50 μL formamide (three times). Load 50 μL of solution (12.4 μL 2-Bromoisobutyryl Bromide, 17.4 μL Diisopropylethylamine, and 970 μL Formamide); sit at room temperature for 15 minutes; repeat twice more.

ii. Load 50 μL 4 M Thiol-containing oligonucleotide (HS-P); sit at room temperature overnight. Wash with water thoroughly.

iii. In-house Quality Control

Hybridize the modified surface with dye-labelled complement oligonucleotide, Fam-Primer.

Image with a fluorescent scanner.

An imaging result of a large-scale patterned flow cell was shown in FIG. 5. Varied patterns may be seen as white features.

### Example 2

i. Introduce the 2-Bromoisobutyryl group onto APTES-modified surface (patterned flow cells) as mentioned above.

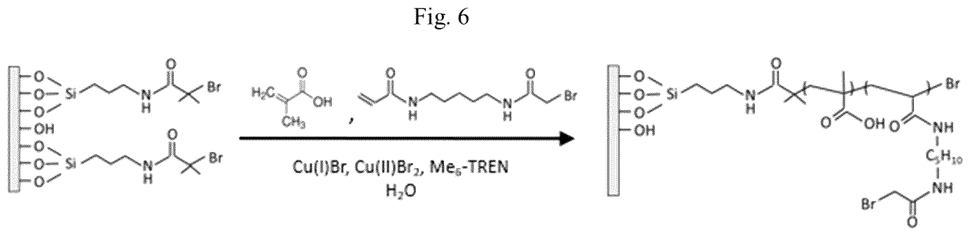

ii. Prepare solution: 763 μL (9.0 mmol) Methacrylic acid, 1.0 mL (10 μmol) Cu(I)Br/Cu(II)Br2/Me6-TREN in water, 2.2 mL water and 1.0 mL (1.0 mmol) Br-monomer (Dimethylformamide) in DMF. Vortex and then centrifuge. Dilute 4-fold with degassed 3.2 mL water with 1.0 mL DMF. Load 800 μL solution through the flow cell with a pump. Sit at room temperature for 2 hours. Wash the flow cell with 3.5 mL water. A reaction principle diagram of preparation of hydrogel was shown in FIG. 6.

iii. Load 50 μL 4 M Thiol-containing oligonucleotide (HS-P); sit at room temperature overnight. Wash with water thoroughly.

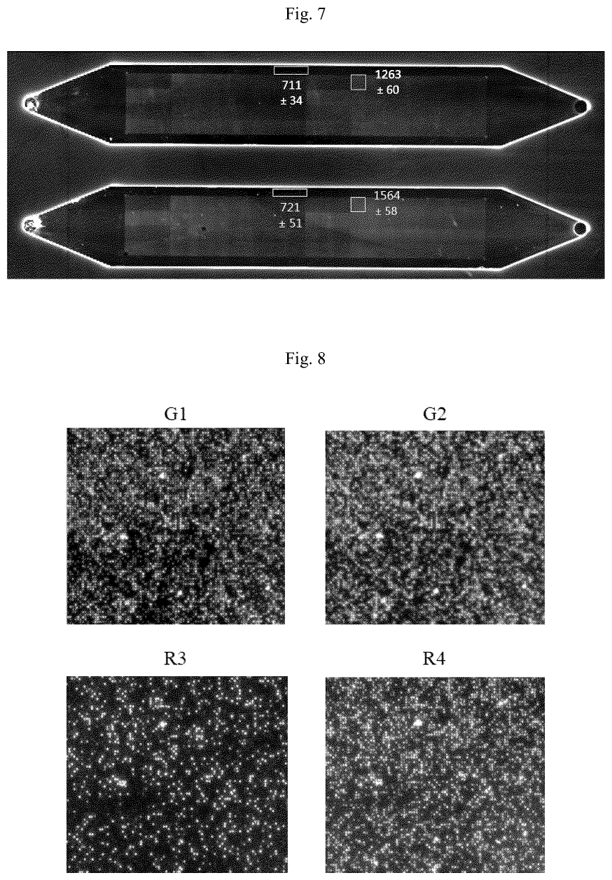

iv. In-house Quality Control
Hybridize the modified surface with dye-labelled complement oligonucleotide, Fam-P′
Image with a fluorescent scanner. An imaging result was shown in FIG. 7.

### 2. Nanoparticle Loading/Capturing

a. Load Nanoparticles from the common Rolling Circle Amplification to a surface-modified flow cell.

b. Cross-link nanoparticles to the features on the patterned surface with UV light (365 nm) for 10 seconds.

c. Wash away non-cross-linked nanoparticles from the solution.

d. Image with a fluorescent microscope or a sequencer.

An imaging result was shown in FIG. 8.

Nanoparticles with four different colors from four different dyes may be observed with four channels (G1, G2, R3, and R4). The nanoparticles may be clearly observed with the composite images.

### Example 3

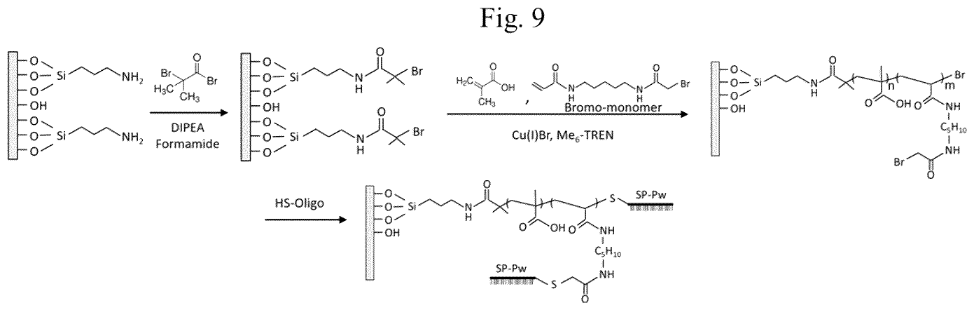

Another feasible method for preparing hydrogel and a method for binding the hydrogel and a first nucleic acid were shown as follows. A reaction principle diagram was shown in FIG. 9.

0.1 M a-Bromoisobutyryl Bromide/0.1 M DIPEA in formamide is reacted with the amino group on patterned flow cells for 15 minutes at room temperature.

Add 806 μL (9.5 mmol) Methacrylic acid, 500 μL (0.5 mmol) Br-Monomer in DMF (1.0 M solution), 1.43 mg (10 μmol) Cu(I)Br and 2.68 μL (10 μmol) Me6-TREN into 3.7 mL of Milli-Q water. Vortex the reaction mixture vigorously for 10 minutes. Load the mixture to the flow cells.

Load 2 mM Thiol-containing oligonucleotide (e.g., SP-Pw) to attach oligonucleotides to the surface of patterned flow cells.

From the above descriptions, it may be seen that, the embodiments of the present application implement the following technical effects: with the method for preparing a patterned flow cell, the reaction reagent (e.g., halogenated acetylation reagent or organic phosphorus compound) reacts with the binding regions that are arranged on the flow cell substrate based on patterns, then the linker is prepared to the hydrogel and then connected with the first nucleic acid, or the linker is directly connected with the first nucleic acid, so as to use the first nucleic acid to capture the microspheres, such that the microspheres are bound with pattern regions of the flow cell substrate to obtain the patterned flow cell. The patterned flow cell can be directly used for subsequent high throughput sequencing, and steps required in the method are less; and the reaction efficiency of each step is high, and the patterned flow cell can be prepared rapidly and inexpensively.

The above are only the preferred embodiments of the present application and are not intended to limit the present application. For those skilled in the art, the present application may have various modifications and variations. Any modifications, equivalent replacements, improvements and the like made within the spirit and principle of the present application all fall within the scope of protection of the present application.

1. A method for preparing a patterned flow cell, comprising:
a) providing a flow cell substrate, wherein the flow cell substrate comprises binding regions, and the binding regions are arranged in a patterned manner on the flow cell substrate;
b) reacting a reaction reagent with the binding regions to obtain a substrate bound with a linker, wherein the linker comprises a halogen atom from the reaction reagent; and
c) connecting the substrate bound with the linker with a first nucleic acid to obtain the patterned flow cell.

2. The method according to claim 1, wherein the reaction reagent comprises a first binding functional group, each of the binding regions comprises a second binding functional group that is used to react with the first binding functional group, the first binding functional group and the second binding functional group are each independently selected from the group consisting of an active ester group, a sulfonyl halide group, an iodoacetyl group, an alkyne group, an amino group, a thiol group, or an azido group;
the reaction reagent further comprises a third binding functional group that is used to connect with the first nucleic acid, and the first nucleic acid comprises a fourth binding functional group that is used to connect with the third binding functional group; or the reaction reagent comprises a halogenated acetyl group that is used to initiate Atom Transfer Radical Polymerization (ATRP);
the third binding functional group comprises the halogen atom; the third binding functional group and the fourth binding functional group are respectively selected from the sulfonyl halide group and the amino group, or the third binding functional group and the fourth binding functional group are respectively selected from the halogenated acetyl group and the thiol group; and
the first binding functional group and third binding functional group in the reaction reagent are different.

3. The method according to claim 1, the method further comprises: d) connecting the patterned flow cell with a microsphere after the patterned flow cell is obtained, wherein
the microsphere comprises a second nucleic acid that is able to bond to the first nucleic acid.

4. The method according to claim 1, wherein the reaction reagent comprises a halogenated acetylation reagent, and the halogenated acetylation reagent comprises the halogen atom;
the binding region is the flow cell substrate connected with a first group; and
the b) comprises:
reacting the binding region comprising the first group with the halogenated acetylation reagent to form the linker, and obtaining the substrate bound with the linker, wherein
the first group has a structure shown in Formula I, and the halogenated acetylation reagent has a structure shown in Formula II; 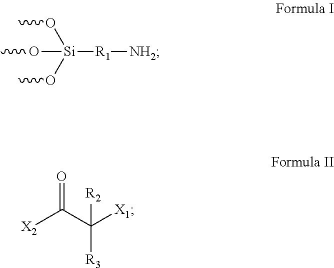
correspondingly, the linker has a structure shown in Formula III, 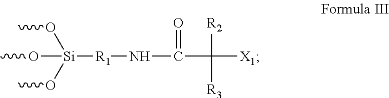
X₁ and X₂ are each independently selected from the halogen atom;
Si is a silicon atom,
the silicon atom is connected with the flow cell substrate through an oxygen atom,
R₁ is selected from alkylidene consisting of 1-4 carbon atoms;
R₂ and R₃ are each independently selected from H, alkyl, or aryl;
preferably, the halogen atom is independently selected from Cl, Br, or I;
preferably, the halogenated acetylation reagent comprises α-Bromoisobutyryl bromide or Bromoacetyl bromide;
preferably, a method for preparing the binding region comprises: contacting a silane with a partial region on the flow cell substrate to obtain the binding region, wherein the silicon atoms in the first group and the linker are all from the silane; and
more preferably, the silane comprises (3-Aminopropyl)triethoxysilane (APTES) or 3-Aminopropyl) trimethoxysilane (APTMS).

5. The method according to claim 3, wherein after the substrate bound with the linker is obtained and before mixing with the first nucleic acid, the substrate bound with the linker is mixed with a polymeric monomer and a catalyst and is subjected to ATRP to obtain a substrate bound with a hydrogel, and then the substrate bound with the hydrogel is bound to the first nucleic acid to obtain the patterned flow cell;
preferably, the polymeric monomer comprises methacrylic acid, acrylamide, N,N-dimethylacrylamide, methyl acrylate, or methyl methacrylate;
preferably, the catalyst comprises a copper atom; and more preferably, the catalyst comprises cuprous bromide.

6. The method according to claim 3, wherein the c) comprises:
the first nucleic acid comprising the thiol group (—SH), and
the thiol group substituting the halogen atom in the linker to obtain the patterned flow cell.

7. The method according to claim 1, wherein the reaction reagent comprises an organic phosphorus compound, and the organic phosphorus compound comprises a phosphonic acid group and the halogen atom;
the binding region comprises a metallic oxide; and
the b) comprises: reacting the metallic oxide with the organic phosphorus compound to form the linker, wherein
the linker has a structure shown in Formula IV, 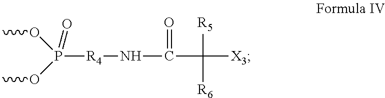
X₃ is the halogen atom;
P is a phosphorus atom from the organic phosphorus compound,
the phosphorus atom is connected with the metallic oxide through an oxygen atom,
R₄ is selected from alkylidene consisting of 1-4 carbon atoms;
R₅ and R₆ are each independently selected from H, alkyl, or aryl;
preferably, the halogen atom is independently selected from Cl, Br, or I; and
preferably, the organic phosphorus compound comprises 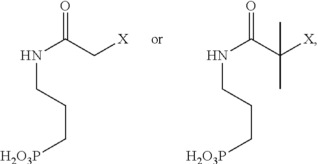
wherein X is Cl, Br, or I.

8. The method according to claim 7, wherein after the substrate bound with the linker is obtained and before mixing with the first nucleic acid, the substrate bound with the linker is mixed with a polymeric monomer and a catalyst and is subjected to ATRP to obtain a substrate bound with a hydrogel, and then the substrate bound with the hydrogel is bound to the first nucleic acid to obtain the patterned flow cell;
preferably, the polymeric monomer comprises methacrylic acid, acrylamide, N,N-dimethylacrylamide, methyl acrylate, or methyl methacrylate;
preferably, the catalyst comprises a copper atom; and more preferably, the catalyst comprises cuprous bromide.

9. The method according to claim 7, wherein the c) comprises:
the first nucleic acid comprising the thiol group (—SH), and
the thiol group substituting the halogen atom in the linker to obtain the patterned flow cell.

10. The method according to claim 7, wherein the metallic oxide comprises one or more of aluminum oxide (Al₂O₃), tantalum oxide (Ta₂O₅), niobium oxide (Nb₂O₅), zirconium oxide (ZrO₂) or titanium oxide (TiO₂);
preferably, the flow cell substrate comprises a glass material; and
preferably, the flow cell substrate comprises a bare semiconductor substrate or a semiconductor substrate.

11. The method according to claim 1, wherein the patterning comprises array arrangement; and
preferably, the binding region is in a same plane as the flow cell substrate, or
there are protrusions or depressions arranged in an array on the flow cell substrate, and the binding region is located in part or all of the protrusions or the depressions.

12. The method according to claim 3, wherein the d) comprises:
d1) mixing the microsphere comprising the second nucleic acid with the patterned flow cell, wherein the second nucleic acid is connected with the first nucleic acid through a hydrogen bond formed by complementary base pairing, so as to obtain a hybrid microsphere substrate; and
d2) under a light condition or a chemical condition, connecting the second nucleic acid in the obtained hybrid microsphere substrate with the first nucleic acid through a chemical bond, and obtaining the patterned flow cell containing the microsphere after cleaning,
preferably, the light condition comprises an ultraviolet radiation.

13. The method according to claim 3, wherein the microsphere comprises an inorganic material microsphere, an organic polymer microsphere, or a DNA Nanoball (DNB),
both the inorganic material microsphere and the organic polymer microsphere are connected to a first target nucleic acid and the second nucleic acid;
the DNB comprises a second target nucleic acid and the second nucleic acid; and
preferably, the inorganic material microsphere comprises an alloy, a metallic oxides, or a silicon oxide.

14. A patterned flow cell, prepared by the method for preparing the patterned flow cell according to claim 1.

15. A patterned flow cell, wherein a substrate of the patterned flow cell is a flow cell substrate, the flow cell substrate comprises connection clusters arranged in an array, each connection cluster comprises a plurality of connection chains, and each connection chain comprises a first nucleic acid and a linker; in a direction away from the substrate of the patterned flow cell, the flow cell substrate, the linker, and the first nucleic acid are connected in sequence.

16. The patterned flow cell according to claim 15, the patterned flow cell further comprises a microsphere, wherein a second nucleic acid is provided on the microsphere, and the second nucleic acid is connected with the first nucleic acid;
the second nucleic acid, the first nucleic acid, and the linker form a connection structure, and
the microsphere and the connection structure form a microsphere chain, so as to connect the microsphere on the flow cell substrate; and
the microsphere further comprises a target nucleic acid.

17. The patterned flow cell according to claim 16, wherein the microsphere chain is in a same plane as the flow cell substrate, or
there are protrusions or depressions arranged in an array on the flow cell substrate, and the microsphere chain is located in part or all of the protrusions or the depressions;
preferably, the microsphere comprises an inorganic material microsphere, an organic polymer microsphere, or a DNA Nanoball (DNB),
both the inorganic material microsphere and the organic polymer microsphere are connected to a first target nucleic acid and the second nucleic acids;
the DNB comprises a second target nucleic acid and the second nucleic acid; and
preferably, the inorganic material microsphere comprises an alloy, a metallic oxides, or a silicon oxide.

18. The patterned flow cell according to claim 16, wherein
in the connection structure, the linker and the first nucleic acid are directly connected through a chemical bond, or the linker forms a hydrogel and then is connected with the first nucleic acid through the hydrogel;
preferably, the first nucleic acid and the second nucleic acid are connected through a hydrogen bond formed by complementary base pairing, or connected through a chemical bond formed by cross-linking under a light condition or a chemical condition;
preferably, the linker has a structure shown in Formula III, 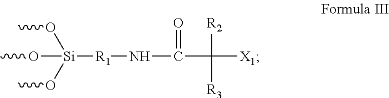
the linker is connected with the flow cell substrate through an oxygen atom; or
the linker has a structure shown in Formula IV, 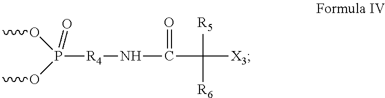
the linker is connected with a metallic oxide on the flow cell substrate, wherein
X₁ or X₃ is each independently selected from a halogen atom, R₁ or R₄ is each independently selected from alkylidene consisting of 1-4 carbon atoms, and R₂, R₃, R₅, or R₆ is each independently selected from H, alkyl, or aryl;
preferably, the halogen atom is independently selected from Cl, Br, or I; and
preferably, the flow cell substrate comprises a glass material;
preferably, the flow cell substrate comprises a bare semiconductor substrate or a semiconductor substrate; and
preferably, the metallic oxide comprises one or more of aluminum oxide (Al₂O₃), tantalum oxide (Ta₂O₅), niobium oxide (Nb₂O₅), zirconium oxide (ZrO₂) or titanium oxide (TiO₂).

19. A sequencing method, comprising sequencing a target nucleic acid with the patterned flow cell according to claim 16.

20. The sequencing method according to claim 19, wherein the sequencing method comprises sequencing by synthesis (SBS),
preferably, the sequencing method comprises:
S1) annealing a sequencing primer onto the target nucleic acid in the microsphere, and extending the sequencing primer with a polymerase and a nucleoside triphosphate, so as to generate a sequencing signal; and
S2) analyzing the sequencing signal, so as to determine sequences of the target nucleic acid in the plurality of microspheres on the patterned flow cell.
